(19) United States
(12) Patent Application Publication
(10) Pub. No.: US 2019/0275177 A1
(43) Pub. Date: Sep. 12, 2019

(54) IDENTIFICATION AND VISUALIZATION OF MICROGRAFTS FOR MONITORING EPITHELIALIZATION

(71) Applicant: KCI Licensing, Inc., San Antonio, TX (US)

(72) Inventors: Kathleen L. DERRICK, San Antonio, TX (US); Diwi L. ALLEN, San Antonio, TX (US); Kristine M. KIESWETTER, San Antonio, TX (US); Deepak V. KILPADI, San Antonio, TX (US); Sandra GARCIA-OSBORNE, San Antonio, TX (US)

(21) Appl. No.: 16/334,160

(22) PCT Filed: Nov. 29, 2017
(86) PCT No.: PCT/US2017/063595

§ 371 Date: Mar. 18, 2019

Related U.S. Application Data
(60) Provisional application No. 62/427,626, filed on Nov. 29, 2016.

(51) Int. Cl. A61K 49/00; A61K 35/36; A61L 27/36

(52) U.S. Cl. CPC A61K 49/0097; A61L 27/362; A61K 35/36; A61K 49/0017

(57) ABSTRACT

An apparatus and methods for distinguishing outgrowth of cells from surrounding biological material are disclosed. In some embodiments, the method may include delivering a marker to cellular material originating from a donor tissue site, followed by transplanting the cellular material from the donor tissue site to a recipient tissue site. One or more locations of the transplanted cellular material at the recipient site may be identified by locating a distribution of the marker at the recipient tissue site.

### CROSS-REFERENCE TO RELATED APPLICATIONS

The present invention claims the benefit, under 35 USC 119(e), of the filing of U.S. Provisional Patent Application Ser. No. 62/427,626, entitled “Identification and Visualization of Microdomes/Epithelialization Versus Slough,” filed Nov. 29, 2016, which is incorporated herein by reference for all purposes.

### TECHNICAL FIELD

The present technology relates generally to tissue treatment systems and more particularly, but without limitation, to devices and methods for cultivating skin graft tissue.

### INTRODUCTION

Skin is the largest organ of the human body, representing approximately 16% of a person's total body weight. Because it interfaces with the environment, skin has an important function in body defense, acting as an anatomical barrier from pathogens and other environmental substances. Skin also provides a semi-permeable barrier that prevents excessive fluid loss while ensuring that essential nutrients are not washed out of the body. Other functions of skin include insulation, temperature regulation, and sensation. However, skin tissue may be subject to many forms of damage, including burns, trauma, disease, and depigmentation (e.g., vitiligo).

Skin grafts are often used to repair such skin damage. Skin grafting is a surgical procedure in which a section of skin is removed from one area of a person's body (autograft), removed from another human source (allograft), or removed from another animal (xenograft), and transplanted to a recipient site of a patient, such as a wound site. Typically it is preferable to use an autograft instead of an allograft or a xenograft to reduce complications, such as graft failure and rejection of the skin graft.

A variety of techniques are known in the art for harvesting and implanting skin grafts. For example, techniques have been developed for harvesting and implanting a large number of small grafts, e.g., micrografts. Such micrografts offer several benefits relative to other techniques among those known in the art, including the reduction of trauma at the donor site by removing only a fraction of the skin traditionally required at the donor site. Certain forms of micrografting leave regions of healthy skin surrounding the excised regions of the donor site, allowing the donor site to heal quickly and reducing the risk of infection.

During the wound healing process, non-viable tissue and tissue debris, or “slough” or “fibrinous slough,” may be generated. This non-viable tissue can be a nidus for infectious agents and must be removed in order to aid in the healing process. Various techniques may be used to remove slough, such as surgical debridement, mechanical debridement, and chemical debridement. The whitish, matte, fibrinous slough material, however, can appear remarkably similar to the early tongues of desirable epithelium which may extend from the graft onto the granulating wound bed. Micrografts, which may include multiple, small sized grafts spread across the implant site, can be particularly difficult to distinguish from slough. Given the similarity in appearance, there is an interest in techniques that could allow differentiation between the slough and healthy tissue so that healing tissue is not inadvertently removed from the wound bed. Accordingly, there is a need to improve the ability to identify and distinguish healing tissue from slough, particularly during the early post-implantation timeframe following graft placement.

### BRIEF SUMMARY

New and useful systems, devices, and methods for harvesting and implanting tissue grafts, including methods and devices useful for identifying viable cellular material, are set forth in the appended claims and the following summary and description. Illustrative embodiments are also provided to enable a person skilled in the art to make and use the claimed subject matter.

Methods are described herein for treating wounds, including epithelial tissue wounds, comprising harvesting donor epithelial tissue comprising cells and a cellular matrix; contacting the donor epithelial tissue with a marker that binds to one or both of the cells and cellular matrix; and implanting the donor epithelial tissue onto a surface of the tissue wound. Such methods may further comprise visualizing the marker on the surface of the tissue wound to identify regions on the surface wherein the donor tissue is implanted. Slough may be removed from the surface of the wound, by removing the slough from regions on the surface other than the regions wherein the donor tissue is implanted, i.e., in regions other than where the marker is visualized. In various embodiments, harvesting comprises the production of micrografts at a donor tissue site. The micrografts may be adhered to an adhesive drape, and then contacted to the tissue wound so as to facilitate migration of the micrograft cells to the wound site.

In various embodiments, markers useful herein bind to either or both of the cells or extracellular matrix in the donor tissue. For example, the marker may bind to a receptor on the cells. Markers may be detected by visible or ultraviolet light in some embodiments. For example, the markers may be fluorescent. In various embodiments, markers are selected from the group consisting of fluorescein isothiocyanate (FITC), fluorescent particles, methylene blue, erythrosine B, ponceaux, and alura red, alcian blue, brilliant blue G, calcein blue, cardio green, crystal violet, fluoroMax, india ink, methyl green, oil red, tattoo ink, quantum dots, picric acid, carbon nanotubes, fuchsins, trichromes. In some embodiments, the fluorescent particles may be polystyrene microspheres.

In some embodiments, methods for distinguishing implanted tissue (e.g., micrografts with outgrowing cells) from surrounding biological material comprise delivering a marker to donor tissue, containing cellular material, transplanting the donor tissue to a recipient tissue site, and locating the donor tissue at the recipient tissue site by detecting the marker at the recipient tissue site. In various embodiments, the donor tissue is skin, comprising epithelial cells. The donor tissue may be transplanted using an adhesive drape.

The present technology also provides an apparatus for differentiating between cellular outgrowth of transplanted micrografts and slough at a tissue site. Such apparatus may include an adhesive drape adapted to transfer one or more micrografts from a donor tissue site to a recipient tissue site and a marker adapted to adhere to the one or more micrografts.

Methods for identifying transplanted micrografts at a tissue site are also described herein, wherein some example embodiments may include applying labeled or fluorescent polymer microspheres upon a surface of an adhesive drape, placing the surface of the adhesive drape in contact with the micrografts, applying the surface of the adhesive drape to the tissue site, and directing a light source onto the tissue site to visualize a location of the micrografts. In some example embodiments, the light source may be a blue LED light or a fluorescent light.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1a is a schematic, perspective view of an example embodiment of a skin micrograft harvesting device in accordance with this specification;

FIG. 1b is a schematic, perspective top view of the skin micrograft harvesting device of FIG. 1a with a head component removed and a cutter mechanism exposed;

FIG. 2a is a schematic, perspective top view of the skin micrograft harvesting device of FIG. 1a with a transfer substrate deployed in the harvesting device to capture skin micrografts;

FIG. 2b is a schematic, side view of the transfer substrate of FIG. 2a;

FIG. 2c is a schematic, perspective bottom view of the transfer substrate of FIG. 2a;

FIG. 2d is a schematic, perspective bottom view of the transfer substrate of FIG. 2a, shown with a plurality of harvested micrografts;

FIG. 3a is a schematic, perspective top view of the transfer substrate of FIG. 2a, shown with a plurality of markers applied;

FIG. 3b is a perspective view of an example embodiment of the transfer substrate of FIG. 2a, shown with a plurality of markers, applied to a tissue site;

FIG. 3c is a perspective view of the tissue site of FIG. 3b, showing the surface of the skin at the tissue site following removal of the transfer substrate of FIG. 3b;

FIG. 4 is a flow diagram of an exemplary process for distinguishing between outgrowth of cellular material from transplanted micrografts and surrounding biological material;

FIG. 5a is an image of a skin donor site stained with a visible marker, according to an example illustrative embodiment;

FIG. 5b is an image depicting skin micrografts stained with the visible marker of the example illustrative embodiment of FIG. 5a;

FIG. 5c is an image depicting harvested skin micrografts stained with the visible marker of the example illustrative embodiment of FIGS. 5a-5b;

FIG. 6a is an image of a skin donor site stained with a fluorescent marker, according to an example illustrative embodiment;

FIG. 6b is an image depicting skin micrografts stained with the fluorescent marker of the example illustrative embodiment of FIG. 6a;

FIG. 6c is an image depicting harvested skin micrografts stained with the fluorescent marker of the example illustrative embodiment of FIGS. 6a-6b; and

FIG. 7 is an image of samples of harvested keratinocytes stained with quantum dots (QDs) of various concentrations and visualized under ultraviolet light, according to another example illustrative embodiment.

### DESCRIPTION OF EXAMPLE EMBODIMENTS

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. In particular, the following description sets forth example embodiments and otherwise provides information that enables a person skilled in the art to make and use the subject matter set forth in the appended claims, but may omit certain details already well-known in the art. The following description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

In various embodiments, the present technology provides methods for wound healing at a tissue site. Such methods generally comprise harvesting a donor tissue from a donor tissue site, and implanting the donor tissue at the site of the wound. The term “tissue site” in this context broadly refers to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), donor sites of flaps and grafts, and/or sites of graft loss, for example. The term “tissue site” may also refer to areas of any tissue that are not necessarily wounded or defective, but are instead areas in which it may be desirable to add or promote the growth of additional tissue.

### Harvesting and Implanting

Methods of the present technology comprise harvesting tissue from a tissue donor site. In various embodiments, the tissue is skin tissue, comprising epidermal cells and their associated extracellular matrix. Such cells may include keratinocytes, melanocytes, Langerhans cells, epithelial stem cells, merkel cells, or combinations thereof.

The term “harvesting” as used herein is intended to encompass the removal of one or more skin grafts by a skin graft generating device. Such devices include those known in the art for obtaining donor tissue. In various embodiments, as further described below, harvesting methods and devices include a suction blister micrograft generator, as well as the transplantation of such skin grafts and any intermediate steps, such as culturing, expanding, stretching, treating, or otherwise preparing a skin graft for transfer to a recipient site. Harvesting of skin grafts can be accomplished in many different ways.

In various embodiments, the donor tissue comprises micrografts. The term “micrograft” as used herein is intended to encompass a skin graft that may be an excised skin segment having at least one dimension parallel to the skin surface that is between 0.5 and 100 millimeters. For example, in some embodiments, a micrograft may be generally circular, oval, or oblong in a plane parallel to the skin surface and may have a diameter or major axis that ranges from about 0.5 mm to 100 mm. Additionally, micrografts also typically have a thickness or depth dimension that extends through the epidermis to encompass basal cells. For example, the depth can range from about 0.01 micrometers to about 500 micrometers, depending on the anatomical location.

Micrografts may be created using devices and methods among those known in the art. For example, one common technique for harvesting a skin graft involves the application of suction to separate a surface portion of the skin, e.g., the epidermis containing the basal cell layer, away from the underlying dermis at the dermal/epidermal (DE) junction. Harvesting of suction blisters typically also involves a heat source to facilitate blister formation.

The application of the suction forces during harvesting of the blisters may be provided by a negative-pressure source. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. Similarly, references to increases in negative pressure typically refer to a decrease in absolute pressure, while decreases in negative pressure typically refer to an increase in absolute pressure. A negative-pressure supply may be a reservoir of air at a negative pressure, or may be a manual or electrically-powered device that can reduce the pressure in a sealed volume, such as a vacuum pump, a suction pump, a wall suction port available at many healthcare facilities, a micro-pump, suction cups, or syringes, for example.

Various devices are commercially available for generating and harvesting micrografts. For example, the CELLUTOME™ Epidermal Harvesting System is available from Acelity L.P., Inc. of San Antonio, Tex. The CELLUTOME™ system may be used in methods of the present technology to harvest epidermal skin grafts consistently, reliably, and with an automated and relatively painless methodology. These micrografts may be comprised of epidermal cells, including but not limited to keratinocytes and melanocytes, which play a critical role in re-epithelialization and re-pigmentation, respectively.

The CELLUTOME™ system harvests many epidermal micrografts, also called microdomes or microblisters. These microdomes may be transferred onto the intended wound/recipient site(s). Once placed onto the granulated wound bed, the keratinocytes migrate from the microdomes onto the surrounding granulation tissue and begin the process of re-epithelializing the surrounding tissue. This outgrowth is expected to occur in concentric circles from the microdomes and allows the wound to be fully re-epithelialized more quickly than occurs when epithelialization is solely occurring from the wound margins.

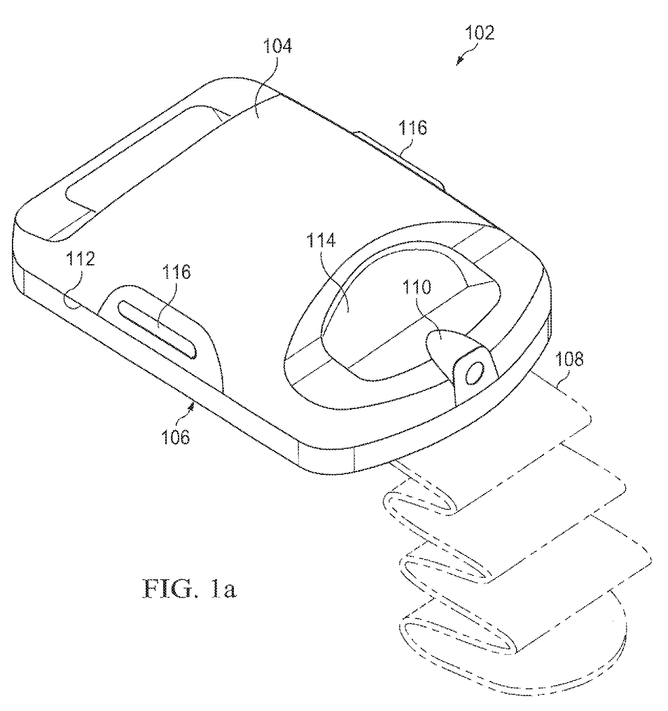

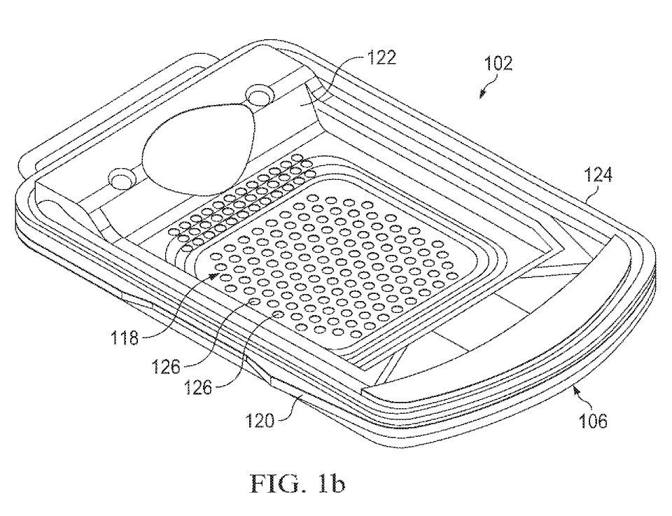

FIG. 1a shows an illustrative embodiment of a skin graft harvester 102 in accordance with various aspects of the present teachings. In this illustrative embodiment, the skin graft harvester 102 may include a detachable head portion 104 and a harvester body 106. The harvester body 106 may be adapted for placement on a patient's skin at a donor site, which may be any anatomical location from where skin grafts are to be obtained, for example, on the inner thigh, and may be secured in place, for example, with strap 108 (shown in phantom). The head portion 104 may further include a heater (not shown) powered via a coupler 110 adapted to couple with a power source in a base unit (not shown). The head portion 104 may further include a seal 112, which may permit a negative pressure chamber to be formed when the head portion 104 and the harvester body 106 are joined together and the skin graft harvester 102 is coupled to a vacuum pump or other source of negative pressure, e.g., via coupler 110 connecting the skin graft harvester 102 to its base unit. The head portion 104 may further include one or more windows 114 for observation of skin blisters being formed within the chamber by application of negative pressure, heat, or both. Once the blisters have been formed, the head portion 104 can be removed, e.g., by deactivating the source of negative pressure and by actuation of release levers 116, which break the seal 112 and allow the head portion 104 to be lifted off of the harvester body 106.

Additional details on harvesters useful in connection with the present invention can be found in U.S. Patent Application Publication No. US 2013-0204273, Sabir et al., published Aug. 8, 2013; U.S. Pat. No. 8,978,234, Sabir et al., issued Mar. 17, 2015; U.S. Pat. No. 9,173,674, Sabir et al., issued Nov. 3, 2015; U.S. Patent Application Publication No. US 2012-0197267, Sabir et al., published Aug. 2, 2012; U.S. Pat. No. 8,562,626, Sabir et al., issued Oct. 22, 2013; U.S. Pat. No. 8,617,181, Sabir et al., issued Dec. 31, 2013; U.S. Pat. No. 8,926,631, Sabir et al., issued Jan. 6, 2015; and U.S. Patent Application Publication No. US 2012-0035619, Sabir et al., published Feb. 9, 2012. The contents of each of the above-referenced related applications are herein incorporated by reference in their entireties.

FIG. 1b is a schematic view of the skin graft harvester 102 of FIG. 1a with the head portion 104 removed and a cutting mechanism 118 exposed. The harvester body 106 may include a base portion 120, a sled 122, and an actuator handle 124. The cutting mechanism 118 may include a plurality of plates with initially aligned holes through which skin blisters may be drawn by heat and/or application of suction when the head portion 104 is joined to the harvester body 106 and activated. Once the blisters are formed, they can be cleaved by the cutting mechanism 118. For example, below a top plate, one or more additional plates, e.g., a cutter plate and a bottom plate can be deployed with aligned holes. By actuation (e.g., pulling up) of the actuator handle 124, the sled 122 is caused to move horizontally such that one of the plates below the top plate, e.g., the “cutter plate” (not shown) also moves (because of its linkage to the sled 122), thereby occluding the alignment of holes 126 and cleaving the raised blisters from the donor's skin.

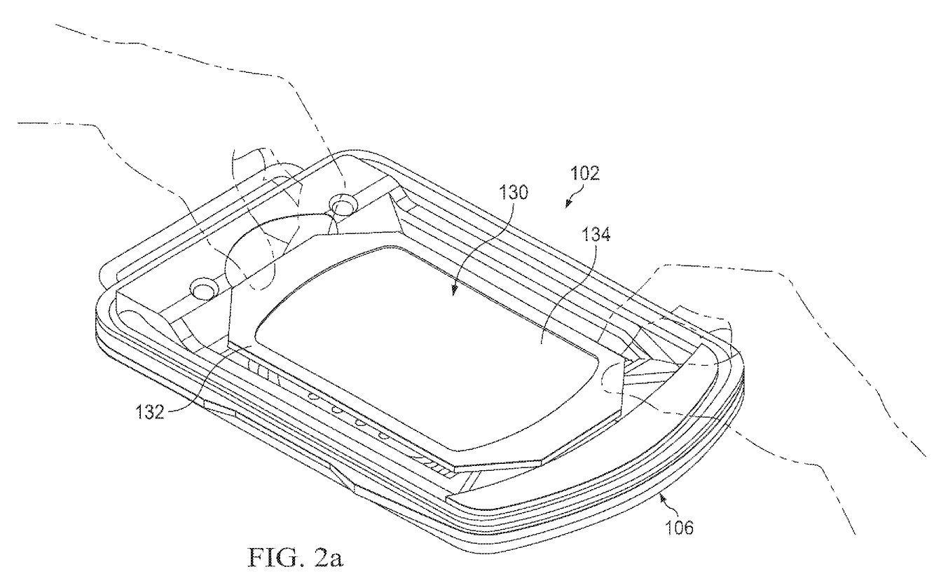

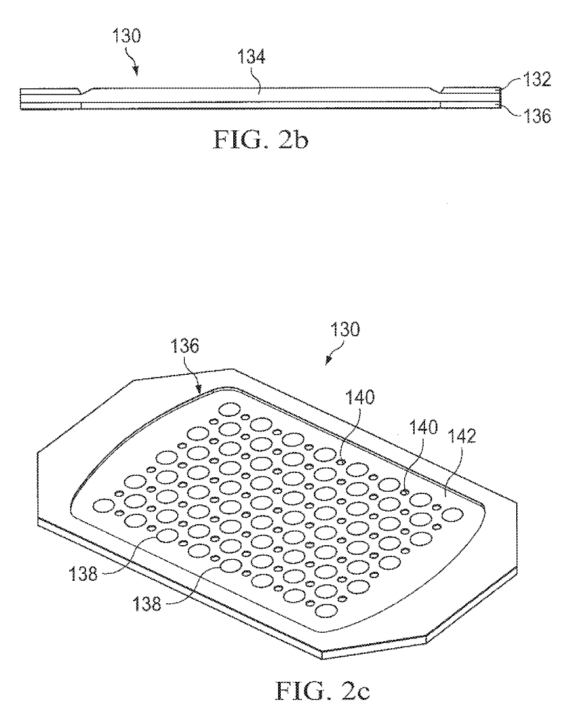

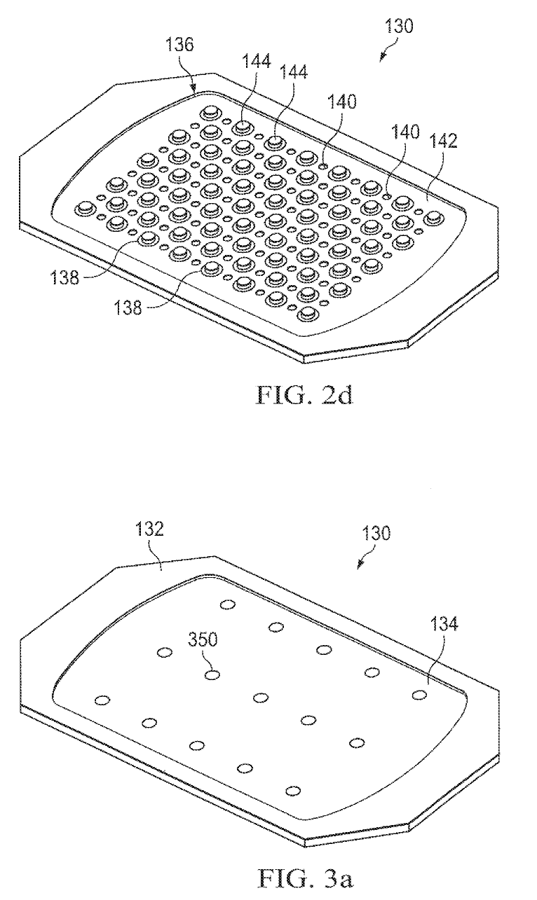

Typically, micrograft harvesters rely upon a support or transfer substrate to lift the excised blisters from the device. As further described below, the transfer substrate is then applied to a recipient site so that the plurality of micrografts can be assimilated as transplanted tissue. FIG. 2a is a schematic view of the skin graft harvester 102 of FIG. 1a with a transfer substrate 130 for capturing skin grafts. The transfer substrate 130 may include a backing 132 for providing structure and support to the transfer substrate 130 and a sealing member 134. In the illustrated embodiment, the user, such as a clinician, may place the transfer substrate 130 in the harvester 102, holding the backing 132. The transfer substrate 130 may also include a base layer (not visible) in contact with the top plate of the cutter mechanism (as shown in FIG. 1b). By such placement of the transfer substrate 130, the base layer may also come into contact with the skin blisters. In one preferred embodiment, the transfer substrate 130 is so situated before the cutter mechanism is actuated to cleave the blisters into skin grafts. In other embodiments, the transfer substrate 130 can be placed onto the harvester 102 after cleavage to capture grafts that have already been cleaved from the skin. In either event, the transfer substrate 130 can then be removed from the harvester body 106 and applied to a recipient site.

Referring now primarily to FIG. 2b, a cross-section view of the transfer substrate 130 of FIG. 2a is shown. Portions of the backing 132, the sealing member 134, and the base layer 136 can be seen. FIG. 2c depicts an embodiment of a transfer substrate, such as transfer substrate 130, viewed from the bottom, showing a base layer 136. The base layer 136 may further include a plurality of graft capture sites 138 for capturing skin grafts, such as micrografts. The entire base layer 136 or just the graft capture sites 138 can be treated, e.g., coated with an adhesive, to make at least portions of the surface of the base layer 136 tacky to promote capture of micrografts. Referring also to FIG. 2d, it can be seen that following cleavage using the skin graft harvester 102, skin grafts, such as micrografts 144, may be adhered to the multiple graft capture sites 138 on the surface of the base layer 136.

The base layer 136 may also be porous, and in this illustrative embodiment, a plurality of pores 140 are disposed between the graft capture sites 138. The pores 140 can be generally circular or elongated in one or more dimensions. Alternatively, the entire surface of the base layer 136 can be porous or can include a network of lines or cross-shaped incisions or openings. Regardless of the shape or size of the pores 140, the porosity of the base layer 136 should be sufficient to permit fluid migration from a skin segment through the base layer 136 for absorption by the transfer substrate 130.

The base layer 136 may have a periphery surrounding a central portion and a plurality of pores 140 disposed through the periphery and/or the central portion. The pores 140 in the base layer 136 may have any shape and may have a uniform pattern or may be randomly distributed on the base layer 136. The diameter of the pores 140 may vary, and in certain embodiments, the average diameter of each of the pores 140 may be between about 0.2 mm to about 2 mm. In some embodiments, the base layer 136 may be fluid permeable, e.g., comprising a material that has high water permeability in either liquid or vapor form, such as polyurethanes, polyesters, polyvinyl chlorides, copolymers of vinyl chloride and vinyl acetate or vinyl chloride and ethylene, polyolefins, polyamides, polyethylene, polypropylene, silicone or polystyrenes, polyacrylics, polyacrylates, polyvinyl alcohol, and copolymers thereof. Examples of water permeable materials include polyurethane films, such as Ensure-IT dressing (Deseret Medical, Inc.) and POLYSKIN® transparent dressing (Kendall Company, Boston, Mass.).

The base layer 136, in some embodiments, may be preferably a soft material suitable for providing a fluid seal with the skin graft transplantation site. For example, the base layer 136 may comprise a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins, polyurethane, polyolefin, or hydrogenated styrenic copolymers coated with an adhesive, as described below. The base layer 136 may have a thickness between about 50 microns (μm) and about 1000 microns (μm). In one embodiment, the base layer 136 has a stiffness between about 5 Shore 00 and about 80 Shore 00. The base layer 136 may include hydrophobic or hydrophilic materials. In some embodiments, the base layer 136 may be a hydrophobic-coated material. For example, the base layer 136 may be formed by coating a mesh or porous material, such as, for example, woven, nonwoven, molded, or extruded mesh with a hydrophobic material, such as a soft silicone.

In some embodiments, the tissue-facing surface of the transfer substrate 130 may include an adhesive 142. The adhesive 142 used to capture skin grafts and/or adhere the transfer substrate 130 to a patient at the transplantation site may be any medically-acceptable adhesive. For example, the adhesive 142 may comprise an acrylic adhesive, rubber adhesive, high-tack silicone adhesive, polyurethane, or other adhesive substance. In some embodiments, the adhesive 142 may be a pressure-sensitive adhesive comprising an acrylic adhesive with a coating weight of 15 grams/m² (gsm) to 70 grams/m² (gsm). The adhesive 142 may be a continuous or a discontinuous layer of material. Discontinuities in the adhesive 142 may be provided by pores 140 in the base layer 136. The apertures in the adhesive 142 may be formed after application of the adhesive 142 to the base layer or by coating the adhesive 142 in patterns on the base layer 136. Factors that may be utilized to control the adhesion strength of the transfer substrate 130 may include the diameter and number of the pores 140 in the base layer 136, the thickness of the base layer 136, the thickness and amount of the adhesive 142, and the tackiness of the adhesive 142. An increase in the amount of the adhesive 142 generally corresponds to an increase in the adhesion strength of the transfer substrate 130. Thus, the size and configuration of the adhesive-coated portions of the base layer 136, the thickness of the base layer 136, and the amount and tackiness of the adhesive 142 utilized may be varied to provide the desired adhesion strength for the transfer substrate 130. For example, the thickness of the base layer 136 may be about 200 microns, and the adhesive 142 may have a thickness of about 30 microns and a tackiness of 2000 grams per 25 centimeter-wide strip.

Referring still to FIGS. 2a-2d, the sealing member 134 may have a periphery and a central portion. The periphery of the sealing member 134 may be positioned proximate the periphery of the base layer 136, such that the central portion of the sealing member 134 and the central portion of the base layer 136 define an enclosure. The sealing member 134 may cover the tissue site to provide a fluid seal and a sealed space between the tissue site and the sealing member 134 of the transfer substrate 130.

The sealing member 134 may be formed from any material that allows for a fluid seal. A fluid seal may be a seal adequate to maintain a positive- or reduced-pressure environment at a desired site given the particular positive- or reduced-pressure source or system involved. The sealing member 134 may comprise, for example, one or more of the following materials: hydrophilic polyurethane; cellulosics; hydrophilic polyamides; polyvinyl alcohol; polyvinyl pyrrolidone; hydrophilic acrylics; hydrophilic silicone elastomers; a thin, uncoated polymer drape; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; polyurethane (PU); EVA film; co-polyester; silicones; a silicone drape; a 3M TEGARDERM™ drape; a polyurethane (PU) drape such as one available from Avery Dennison Corporation of Pasadena, Calif.; polyether block polyamide copolymer (PEBAX), for example, from Arkema, France; or other appropriate material.

The sealing member 134 may allow vapor to exit while inhibiting liquids from exiting the sealed space provided by the transfer substrate 130. The sealing member 134 may be a flexible, breathable film having a high MVTR of, for example, at least about 300 g/m² per 24 hours. The sealing member 134 may comprise a range of medically-suitable films having a thickness between about 15 microns (μm) to about 50 microns (μm). In other embodiments, a low- or no-vapor transfer drape can be used as the sealing member 134.

Methods further comprise implanting the donated tissue at a recipient site, e.g., the site of a wound. As referred to herein, “implanting” refers to any clinically suitable method by which donor tissue is applied on or to a recipient site so as to allow for survival and, preferably in various embodiments, growth of the donor tissue at the recipient site. For example, in further reference to FIGS. 2a-2d, a transfer substrate 130 containing a plurality of harvested micrografts, such as micrografts 144, may be applied to a recipient site of a patient. The size of the area at the recipient site can be larger or smaller, but preferably about the same size as the area of the transfer substrate 130 having micrografts adhered thereto. Prior to applying the grafts to the recipient site, the site can be prepared to receive the grafts using any technique known in the art. Necrotic, fibrotic, or avascular tissue should be removed so that the grafts may be applied to a clean, properly-prepared wound bed. The technique used to prepare the site will depend, for example, on damage to the recipient site. For example, epidermal tissue, if present at the recipient site, can be removed to prepare the area for receiving the micrografts. Burned or ulcerated sites may not need removal of epidermal tissue, although some cleaning of the site or other preparation of the site may be performed. Wounds should be debrided and then allowed to granulate for several days prior to applying the graft. Necrotic tissue should be removed since it has a tendency to harbor bacteria.

The transfer substrate 130 having the plurality of adhered micrografts, such as micrografts 144, may be placed over the area to be treated, such as the recipient site. A portion of the transfer substrate 130 having the attached micrografts can be positioned over the area to be repaired, e.g., the wound area or the area from which the epidermal tissue has been abraded or removed for re-pigmentation. The transfer substrate 130 may be fixed in place over the treatment area, e.g., using tape, a secondary dressing or the like. The transfer substrate 130 may be removed after sufficient time has elapsed to allow attachment and growth of the micrografts in the recipient site, e.g., several days to a few weeks. Once applied to the recipient site, the micrografts may expand and coalesce to complete the healing process. During this timeframe, the extracellular components of the micrografts undergo changes to allow for the cellular outgrowth.

### Markers and Methods of Visualizing Viable Tissue

The present technology provides “markers,” which may be materials that bind to one or more components of donor tissue. In various embodiments, such donor tissue components may include cells and cellular materials including, but not limited to, proteins, all types of ribonucleic acids including mRNA, microRNA, CRISPR RNA etc., deoxyribonucleic acids, lipids, signaling molecules, carbohydrates, and phospholipids. Extracellular materials may include keratin or lipids, collagens, elastins, vitronectins, laminins, fibrillins, fibronectin, integrins, glycosaminoglycans, proteoglycans, lectins, fatty acids, ceramides or other materials secreted by the donor tissue cells. Binding of the marker to the component may be through any chemical or biochemical modality which results in association of the marker with the component such that visualization of the marker at a region of tissue is indicative of the presence of the component at that region, under typical clinical conditions. In some embodiments, a marker is bound to one or more receptors on donor tissue cells. For example, a dye may attach to the cell nuclei or the cellular membranes. It is understood, though, that given the nature of the epidermis, the dyeing of the cells and/or extracellular matrix may not be permanent and may fade or be exfoliated with time. Additionally, the signal of a marker may be attenuated as the dyed cells undergo cell division. In some additional embodiments, a marker may also be ingested by the target cells. In some embodiments, markers may be bound to cells or extracellular matrix components either at the time of harvesting the donor tissue or at the time of evaluating the tissue in question at the recipient site. In some embodiments, the marker may be activated or deactivated by viable cells, which may allow the markers or molecules to be observable by the appropriate imaging modality.

Markers, such as dyes, can be non-specific or specific. Some dyes may interact via diffusion (non-specific) or at different regions of cells (specific). Different dyes or stains may react or concentrate in different parts of a cell or tissue, and these specific properties may be used as a tool for revealing specific parts or areas. Non-specific dyes can enter the most-upper layers of the skin like the epidermis/stratum corneum by diffusion or by being cell-permeable. Dyes such as methylene blue, for example, work by binding to biological tissues through chemical attractions. Methylene blue is at its deepest shade of blue when in contact with acids. This property makes the dye very useful in the identification of nucleic acids, such as DNA and RNA which may be present within all cell types (epidermal cells). As another example, fluorescein isothiocyanate (FITC) may demonstrate conjugation with the following reactive groups: amine (NHS ester, isthiocyanate), thiol (maleimide, iodoacetamide, bromoacetamide), and aldehyde, ketone carboxylate or phosphate (thiosemicarbazide). Lipid dyes may be attached to the fatty acid chains or to the phospholipids themselves. Other dyes may demonstrate very specific binding behavior. For example, antibodies may have an antigen binding site which is located at the top of each of the arms. Each site may be defined by 6 loops called Complementary Determining Regions (CDR). Three of these loops may be found on the heavy chain (H1, H2, and H3) and three on the light chain (L1, L2, and L3). These protein loops may mirror, or complement, the shape of specific antigens. As a result, they may determine to which specific antigens the antibody can and will bind. The dye can then be tailored to the antigen binding site.

Other types of cell-surface molecule biosensors include fluorescent, or visual dyes conjugated to lectins which may be targeted to specific membrane carbohydrates including: Concanavalin A (Con A) isolated from jack bean; Wheat Germ Agglutinin (WGA) isolated from Triticum vulgaris; IB4 isolated from an African legume; GS-II isolated from Griffonia simplicifolia; PHA-L isolated from red kidney bean; HPA isolated from edible snail; SBA isolated from soybean; and PNA isolated from peanuts.

Markers useful herein are preferably non-toxic to the donor tissue and to tissue at the wound site under typical clinical conditions. Such markers may be, for example, dyes used in culturing and histological staining methods among those known in the art. Preferably, markers may be visualized using visible or ultraviolet light. In various embodiments, markers may fluoresce.

As previously mentioned, in various embodiments, the marker may comprise a dye. Suitable dyes may include but are not limited to fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, sudan III, methyl green, oil red, pyronin Y, tattoo ink, purpurin, quantum dots, phloxine B, picric acid, carbon nanotubes, fuchsins, resazurin and trichromes. More specifically, some example applications may include FITC, erythrosine B, ponceau S, brilliant blue and cardio green for visualizing proteins; methylene blue, SYBR green, purpurin, and DAPI for nucleic acid staining; pyronin Y for specifying RNA; oil red, nile blue and sudan III to demonstrate the presence of lipids; alcian blue for staining polysaccharides & glycosaminoglycans; indigo carmine for visualizing collagen; phloxine B as a keratin stain; and resazurin for the measurement of metabolic activity of mitochondria (reduction/oxidation) for indicating cell proliferation and viability.

In some embodiments, as with fluorescent particles, the particular dye may be encapsulated within a polymer microsphere, for example polystyrene microspheres, and therefore, the dye may never be in direct contact with the skin. In some additional example embodiments, the markers may include quantum dots (QDs), which may be illuminated with the application of a light source. QDs may be engineered semiconductor nanomaterials and may penetrate intact skin typically using vertical silicon nanowires, and have great potential as diagnostic and imaging biosensors. QDs may comprise a colloidal core with a number of surface-coated layers. The number of coatings and types of coatings may be utilized for bio-sensing specificity and may include hydrophilic, neutral, anionic or cationic coatings, or coatings with functional surface groups that serve as ligands to specific cell receptors. QDs may be fluorescent, photostable, and commercially-available in various sizes and shapes, for example spherical or ellipsoid. They may also emit at various emission maxima typically ranging from wavelengths of 525-655 nm.

Further embodiments may incorporate the use of different light sources or other stimulus inputs for illuminating or otherwise visualizing a marker associated with the donor tissue. For example, various light sources may include blue LED (max wavelength˜505 nm), red LED (max wavelength˜635 nm), green LED (max wavelength˜570 nm), polarized light, Raman signals, and/or FTIR signals and imaging mechanisms to visualize the potential differences between donor tissue and slough through either direct or indirect means.

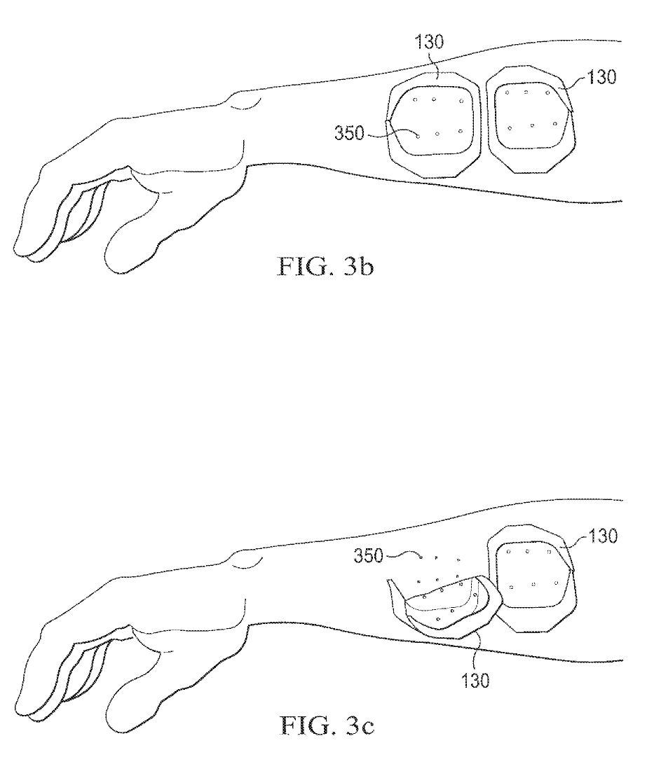

In some embodiments, the present technology provides a tissue transfer substrate useful for differentiating between cellular outgrowth of transplanted micrografts and slough at a tissue site, comprising: an adhesive drape or dressing adapted to transfer one or more micrografts from a donor tissue site to a recipient tissue site; and a marker adapted to adhere or diffuse into the one or more micrografts. FIG. 3a is a schematic view of an embodiment of an exemplary transfer substrate 130 of FIGS. 2a-2c, viewed from the top, showing a backing 132 and a sealing member 134. As discussed above, the sealing member 134 may be substantially transparent, and may serve as a window for observing the harvesting of micrografts from beneath the transfer substrate 130. The sealing member 134 may be suitable for adhering a mechanism for indicating the location of one or more micrografts, such as marker 350. In some example embodiments, the marker 350 may include fluorescent particles that are applied to the sealing member 134, or more generally to the transfer substrate 130, and allowed to dry for a predetermined period of time, which in some instances may be between about 15 minutes to 45 minutes. For example, the sealing member 134 may have an indicator element, such as marker 350, incorporated with it such that upon contact and pressure the marker 350 may be exposed to the micrograft and labels the micrograft and/or surrounding tissue. Thus, when the micrografts are harvested, each individual micrograft may include a slight amount, or dot, of marker 350, such as a dye. The transfer substrate 130 may then be applied to the recipient site and left in place for an amount of time, which in some example embodiments may be for about 30 seconds or less. In embodiments where the transfer substrate 130 includes a base layer 136, the sealing member 134 may at least allow for a user to view the marker 350 that may be adhered to the base layer 136, through the sealing member 134.

Referring now to FIGS. 3b and 3c, during use, the markers 350 may be delivered non-invasively to skin tissue to be grafted. For example, in some example embodiments, the markers 350 may be bound to or incorporated within the transfer substrate 130. In some embodiments, the transfer substrate 130 may be an adhesive drape or dressing, such as a TEGADERM drape, available from 3M, or a silicone dressing, as previously discussed. As illustrated in FIGS. 3b-3c, the transfer substrate 130 may be applied with the tissue-facing surface, such as the base layer 136, against the patient's skin at the recipient well-prepared wound site.

In one example embodiment, the markers may be delivered using polymer microspheres. The polymer microspheres may be used to encapsulate a particular dye, such as but not limited to a fluorescent material. In this embodiment, small dots of the microspheres or nanospheres containing the fluorescent material of various emission maxima may be placed upon the transfer substrate 130, and more specifically on the tissue-facing surface of the base layer 136. The small dots of microspheres may then be allowed to dry. Alternatively or additionally, in some embodiments, the majority of a surface of the transfer substrate 130, such as the tissue-facing surface of the base layer 136, may be coated with such microspheres or nanospheres. As shown in FIG. 3b, the transfer substrate 130 may then be placed onto a patient's wound or recipient site(s) (in the case of FIG. 3b, for illustrative purposes, intact skin). The transfer substrate 130 may then be left in place on the recipient site to allow the micrografts to become incorporated into the underlying tissue site. After an appropriate time whereby the epidermal cells and associated material migrate out from the micrograft onto the adjoining granulation tissue, the transfer substrate 130 may be removed from the recipient site, as shown in FIG. 3c. The time frame for this migration to occur may generally be at least 5-7 days, but may be as short as 3 days, and as long as 14 days. Due to the interaction of the marker 350 with the micrografts, the micrografts may be labeled on the recipient site and may be distinguishable from any fibrinous slough that may have formed within the recipient site. In the context of a wound, the marker 350, such as a dye, may diffuse into the wound bed, such as the membrane of the epidermis/dermis junction, as the cells in the micrograft migrate. When applied to intact skin, as depicted in FIGS. 3b-3c for illustrative purposes, the marker 350 may be diffused into the stratum corneum. The marker 350, such as a dye, in this particular case, may be visualized either by the naked eye or by using a visualization device such as a blue LED light or other similar light device.

The present technology also provides methods for visualizing viable tissue comprising contacting the tissue, e.g., donor tissue, with a marker. In various embodiments, such methods associated with the harvesting of donor tissue and implanting the tissue at a wound site provide methods for identifying the grafted tissue, so that clinicians and patients may be able to better distinguish the viable donor tissue from undesirable slough. Thus, such methods provide a color identification system by which the clinicians can visualize epidermal cellular outgrowth of cells, such as keratinocytes, so as to identify viable tissue that should not be removed during wound debridement.

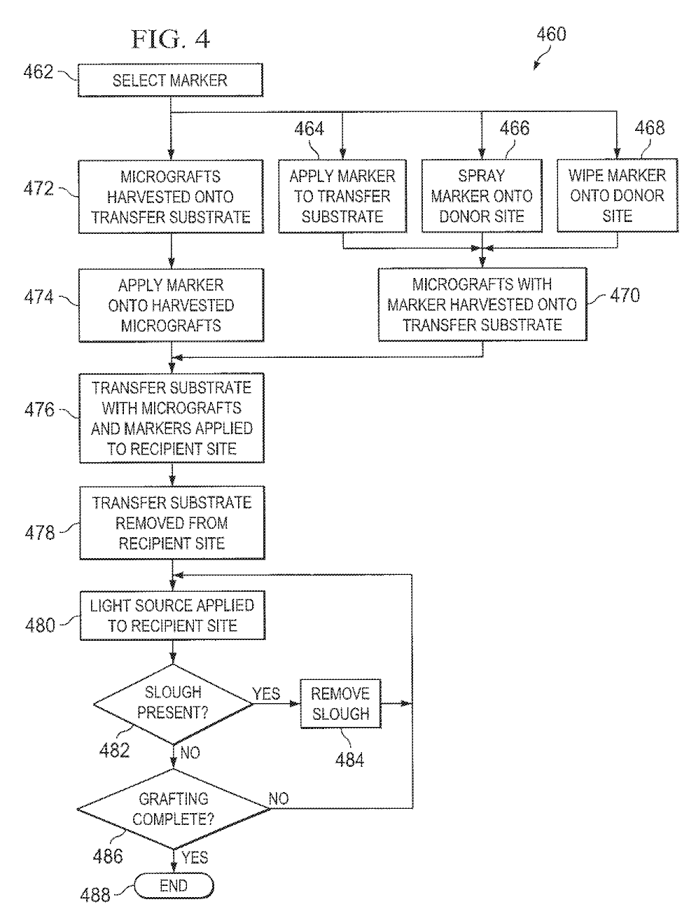

With reference to FIG. 4, an exemplary method 460 for distinguishing between outgrowth of cellular and extracellular material from transplanted micrografts and surrounding biological material is described in conjunction with the use of micrograft harvesting, as described above. However the method 460 may also be useful in other applications of differentiating cellular tissue from surrounding biological material, with or without the use of skin grafts. As shown in step 462, the method 460 for visualizing harvested micrografts may begin with selecting an appropriate marker to be used with the type of tissue being grafted. As previously described, the marker may include both a specific indicator, such as a dye, as well as a means for attachment to the targeted graft tissue. Therefore, multiple considerations may factor into the selection of a particular marker for a given micrograft application.

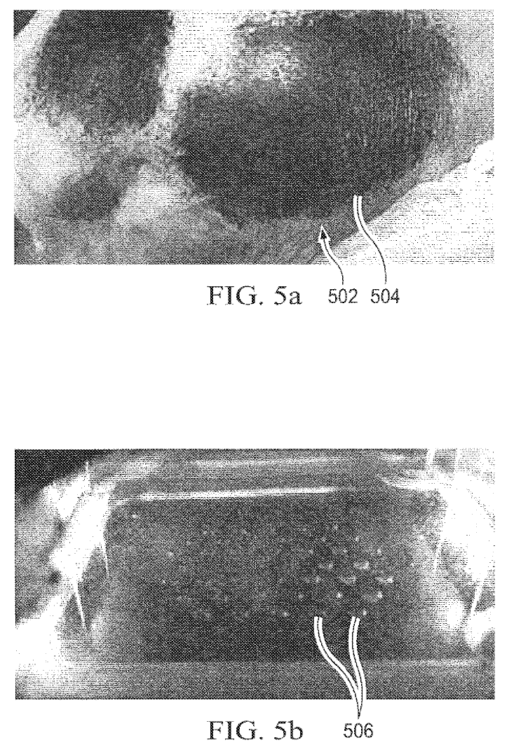

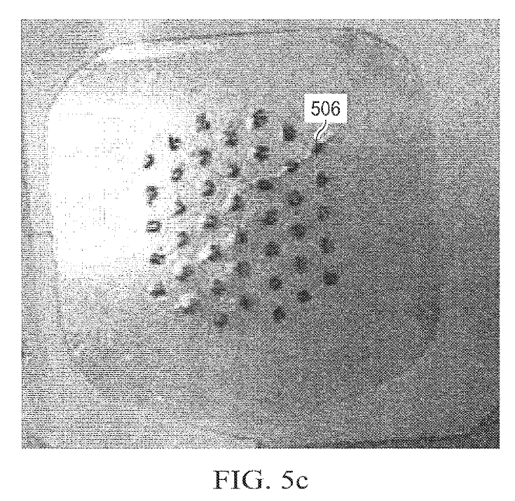

Following selection of the particular marker at step 462, the marker must be applied to the targeted tissue to be grafted. Depending on the specific application, a user, such as a clinician, may choose from a variety of possible means for delivering the marker to the targeted tissue. For example, as depicted in step 464, the marker may be applied to a transfer substrate, such as the transfer substrate 130. In some embodiments, the marker may be bound to or incorporated within the transfer substrate 130. Alternatively or additionally, the clinician may decide to deliver the marker directly to the tissue to be grafted at the donor site. For example, in some embodiments, the marker may be sprayed onto the tissue at the donor site, as shown in step 466. The marker may also be wiped onto the tissue at the donor site, which is depicted in step 468. For example, referring also to FIG. 5a, it can be seen how a skin donor site 502 may be stained with a visible marker 504. Following the application of the marker to either the transfer substrate (step 464) or directly to the donor site (steps 466 or 468), the micrografts may be harvested onto the transfer substrate 130, as shown in step 470. Referring now also to the examples shown in FIGS. 5b-5c, it can be seen that at this stage of the method, the marker should now be incorporated in the micrografts to provide the stained micrografts 506.

The method 460 may also include delaying the application of the marker until after the micrografts have been harvested. For example, following the selection of the marker in step 462, the micrografts may be harvested onto a transfer substrate, such as the transfer substrate 130, as shown in step 472, following which, the marker may then be applied, by soaking, spraying or wiping, onto the harvested micrografts that are attached to the transfer substrate 130, as depicted in step 474.

Once the micrografts, including the marker, have been incorporated onto the transfer substrate 130, the transfer substrate 130 with the micrografts and marker may be applied to the recipient site, as shown in step 476 of the method 460. Following a designated period of time, the transfer substrate 130 may be removed from the recipient site, leaving behind the micrografts and marker, as shown in step 478.

Following removing the transfer substrate 130 from the recipient site, the marker may be visible at the recipient site. Depending on which specific type of dye the marker includes, some of the marker may be visualized by way of the naked eye. For example, dyes including methylene blue and India ink may be seen without the aid of an additional instrument, as may be the case for the dye depicted in FIGS. 5a-5c. Otherwise, the marker may include some dyes that may require the application of a light source, such as a wand light, to visualize the marker and associated micrografts and tissue proliferation from the micrografts, as shown in step 480 of the method 460. For example, if the marker includes the fluorescein isothiocyanate (FITC) dye, ultraviolet light may be applied in order to visualize the micrografts containing or adhered to the marker. In the case of the dye including fluorescent particles, a blue LED light may be administered to visualize the micrografts with the marker. Similarly, a camera capable of emitting known visual, infrared or ultraviolet wavelengths may be used to stimulate the marker and obtain an image to indicate the location/presence of the micrographs.

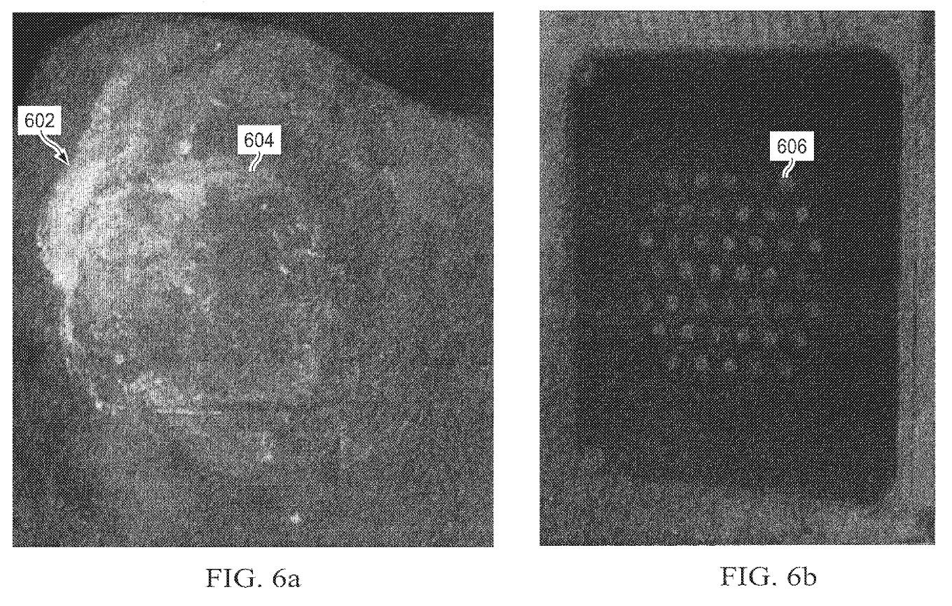

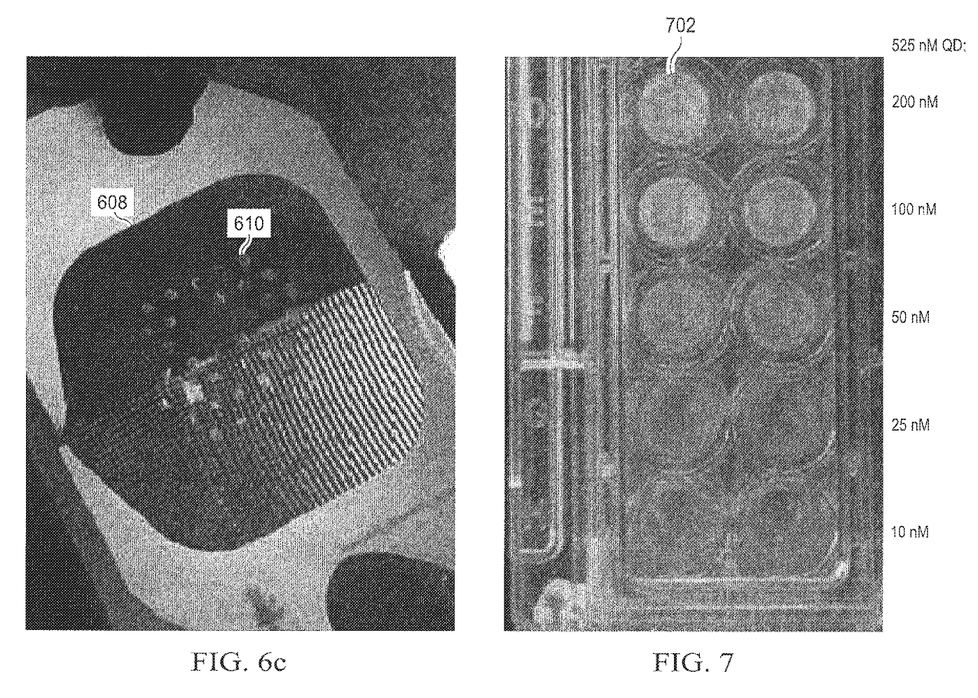

For example, FIG. 6a depicts an example of how a skin donor site 602 may be stained with a fluorescent marker 604. As shown in FIGS. 6b-6c, the resulting harvested micrografts may be stained with the fluorescent marker 604, as shown with the stained micrografts 606 on the transfer substrate 608. Additionally, FIG. 7 illustrates an example of samples of quantum dot-stained keratinocytes 702. Such quantum dots may be biocompatible with keratinocytes at a wide range of concentrations, and may be visible under exposure to ultraviolet light, for example ultraviolet light of 50-200 nm in wavelength.

Referring again primarily to FIG. 4, upon applying the light source, a user, such as a clinician may be able to distinguish between the desirable micrografts and associated cell and tissue proliferation associated with the micrografts and undesirable slough, as shown in step 482 of the method 460. For example, as the light source is applied to the recipient site, the marker, which may include a dye, may not be visible, which may then indicate to a clinician that the material could be slough. Should the clinician determine that slough is present at the recipient site, the slough may then be removed, such as by wiping off of the recipient site, in step 484. Following the removal of the slough in step 484, the clinician may once again apply the light source to the recipient site, according to step 480, in order to determine whether some amount of undesirable matter, such as the slough, is still present at the recipient site.

Should the clinician determine in step 482 that there does not appear to be slough present at the recipient site, the clinician may then make an assessment as to whether the grafting process of the micrografts at the recipient site is completed, according to step 486 of the method 460. In some instances, the grafting process may be considered complete when the clinician determines that the harvested micrografts, such as microdomes, have fully “taken” into the recipient site, in accordance with clinical standards understood in the art and tissue grafting practices. Such a determination may vary based on the size, shape, location, or other factors specific to the recipient site, as well as factors related to the individual patient. Additionally, in some circumstances, the grafting process may be considered to have concluded once the recipient site has fully undergone healing. Should the clinician make the assessment that the grafting is not complete, the recipient site may be periodically reassessed, in accordance with steps 480-486. Alternatively, once it is determined that the grafting is complete, the method may be complete, as depicted in step 488.

In addition to those embodiments already discussed, some embodiments of the present invention may additionally or alternatively include a variety of other features. For example, numerous delivery mechanisms may be used for applying the marker to the micrografts. In some embodiments, the marker may be specifically incorporated within custom features of a transfer substrate 130. For example, the marker may be placed within pressure-sensitive portions of an adhesive of the transfer substrate 130. The markers may then be released with the application of pressure, such as by a clinician pressing against the transfer substrate 130, as the micrografts are being seated onto the transfer substrate 130. In some embodiments, the outside area or border of an adhesive surface of the transfer substrate 130 may be “spotted” with the marker, which may allow for a clinician to identify the border in which the micrografts originate. This “spotting” mechanism may allow a clinician to readily determine where the micrografts first started, which may further provide the clinician with a means for tracking the growth progress of the micrografts over time. This mechanism may also contribute to a cost-savings, as a smaller amount of marker and associated dye material may be applied to the transfer substrate 130.

Additional embodiments may incorporate other methods and mechanisms for differentiating between viable micrograft tissue and slough at a recipient site. For example, areas of tissue at a recipient site that are in question may be evaluated for a range of biomechanical and/or biophysical properties that may provide indications of whether the tissue is viable tissue or slough. Such methods may involve direct contact of the tissue at the recipient site with an appropriate stress- or strain-sensing tool. Such tools could include a measure of physical resistance to a highly-sensitive probe tip applying loads or frequencies of challenge (e.g., a vibrating tip) that are not deleterious to micrografts but provide sufficient challenge to differentiate responses from grafts versus slough. Some methods may also involve the use of ultrasound diagnostics of the tissue area, as well as challenges to the tissue site, such as flexion/relaxation of a limb, temperature challenges, oxygen inhalation or other physical challenges, in conjunction with physiological-response-measuring tools, such as, but not restricted to, thermal imagers/cameras and oxygen detection systems (e.g., hyperspectral imaging). For example, in one example embodiment, a highly-sensitive temperature camera may be used to discern if micrografts were still in place. Using such a camera, the micrografts should show up as dots against the wound background. In another example embodiment, a hyperspectral imaging camera may be used, in which case the area(s) of the wound under the micrografts should present a different oxy/deoxy profile than that of the surrounding wound bed, thus allowing for differentiation between the types of material at the recipient site.

The systems, materials, and methods described herein may provide significant advantages. As previously mentioned, there is a present need in the market for means of differentiating between cellular outgrowth and undesirable material at a tissue site, such as slough. By addressing this outstanding need, the disclosed materials and methods can offer a variety of benefits. For example, by providing a more accurate, reliable, and quicker way for identifying slough at a recipient site, clinicians may (1) refrain from inappropriately removing micrografts that are mistaken for slough and (2) spend less time performing procedures associated with the micrograft recipient site, while allowing for improved outcomes such as faster tissue site closure and re-epithelialization. Clinicians may also be able to more quickly identify if the recipient site is in need of debridement or an alternative procedure due to the presence of slough. Furthermore, the need for repeated micrografting may be reduced, as clinicians will be less likely to unintentionally wipe away or otherwise remove desirable cellular outgrowth from the micrografts, therefore providing a potentially substantial cost savings.

The described inventions may also be applicable outside of the realm of tissue grafting. For example, the disclosed systems, apparatuses, and methods may be used with other types of wounds, for instance, to assist a clinician with understanding which parts of a wound could be debrided or understanding if the edges of a wound at a periwound site are macerated or if new, fresh epithelium is in fact developing.

While shown in a few illustrative embodiments, a person having ordinary skill in the art will recognize that the systems, apparatuses, and methods described herein are susceptible to various changes and modifications. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described herein may also be combined or replaced by alternative features serving the same, equivalent, or similar purpose without departing from the scope of the invention defined by the appended claims.

What is claimed is:

1. A method for distinguishing healthy implanted tissue from surrounding biological material, comprising:
delivering a marker to donor tissue, wherein the marker is adapted to attach to the donor tissue;
implanting the donor tissue to a recipient tissue site;
locating the donor tissue at the recipient tissue site by detecting the attached marker at the recipient tissue site.

2. The method of claim 1, wherein the donor tissue comprises multiple micrografts.

3. The method of claim 2, wherein the micrografts have a thickness in the range of approximately 0.01 to 500 micrometers.

4. The method of claim 2, wherein the micrografts are generally circular in shape and have a diameter in the range of approximately 0.5 to 100 millimeters.

5. The method of claim 2, wherein the micrografts comprise epidermal cells.

6. The method of claim 5, wherein delivering the marker to the donor tissue comprises delivering the marker to the epidermal cells at an epidermal-dermal junction.

7. The method of claim 1, wherein implanting the donor tissue comprises adhering the donor tissue to an adhesive drape and contacting the adhered donor tissue with the recipient tissue site.

8. The method of claim 7, wherein delivering the marker to the donor tissue comprises binding the marker to the adhesive drape.

9. The method of claim 1, wherein delivering the marker to the donor tissue comprises applying the marker to the donor tissue.

10. The method of claim 9, wherein applying the marker to the donor tissue further comprises spraying the donor tissue with the marker.

11. The method of claim 9, wherein applying the marker to the donor tissue further comprises soaking the donor tissue in the marker.

12. The method of claim 9, wherein applying the marker to the donor tissue further comprises wiping the marker onto the donor tissue.

13. The method of claim 8, wherein binding the marker to the adhesive drape further comprises spraying the marker onto the adhesive drape.

14. The method of claim 1, wherein the donor tissue comprises cells and extracellular matrix, and the marker is adapted to bind to at least one of the cells and the extracellular matrix.

15. The method of claim 1, wherein the marker is adapted to bind to a receptor on cells of the donor tissue.

16. The method of claim 1, wherein the marker is adapted to bind to at least one of the following associated with the donor tissue, selected from the group consisting of protein, ribonucleic acid, deoxyribonucleic acid, lipids, signaling molecules, phospholipids, and carbohydrates.

17. The method of claim 16, wherein the ribonucleic acid includes at least one of the following selected from the group consisting of mRNA, microRNA, or CRISPR RNA.

18. The method of claim 5, wherein the epidermal cells comprise at least one of the following selected from the group consisting of keratinocytes, melanocytes, Langerhans cells, epithelial stem cells, and merkel cells.

19. The method of claim 1, wherein the marker is adapted to bind to at least one selected from the group consisting of collagens, elastins, vitronectins, laminins, fibrillins, fibronectin, integrins, glycosaminoglycans, proteoglycans, lipids, lectins, fatty acids, ceramides or other materials secreted by the donor tissue.

20. The method of claim 1, wherein the marker is detected by visual or ultraviolet light.

21. The method of claim 1, wherein the marker is fluorescent.

22. The method of claim 1, wherein the marker is selected from the group consisting of fluorescein isothiocyanate, fluorescent particles, methylene blue, erythrosine B, ponceaux, and alura red, india ink, indocyanine green, alcian blue, brilliant blue G, calcein blue, cardio green, crystal violet, fluoroMax, methyl green, oil red, tattoo ink, quantum dots, picric acid, carbon nanotubes, fuchsins, and trichromes.

23. The method of claim 21, wherein the marker comprises fluorescent particles comprising polystyrene microspheres.

24. The method of claim 16, wherein the marker is specific to proteins within an epidermal layer of the donor tissue site.

25. A tissue transfer substrate useful for differentiating between cellular outgrowth of transplanted micrografts and slough at a tissue site, comprising:
an adhesive drape adapted to transfer one or more micrografts from a donor tissue site to a recipient tissue site;
a marker adapted to adhere to the one or more micrografts.

26. The tissue transfer substrate of claim 25, wherein the marker is detected using visual or ultraviolet light.

27. The tissue transfer substrate of claim 26, wherein the marker comprises fluorescent properties.

28. The tissue transfer substrate of claim 25, wherein the marker is selected from the group consisting of fluorescein isothiocyanate, fluorescent particles, methylene blue, erythrosine B, ponceaux, and alura red, alcian blue, brilliant blue G, calcein blue, cardio green, crystal violet, fluoroMax, india ink, methyl green, oil red, tattoo ink, quantum dots, picric acid, carbon nanotubes, fuchsins, and trichromes.

29. The tissue transfer substrate of claim 28, wherein the marker comprises fluorescent particles comprising polystyrene microspheres.

30. The tissue transfer substrate of claim 25, wherein the marker binds to a cellular receptor.

31. The tissue transfer substrate of claim 25, wherein the marker is adapted to bind to lipids, fatty acids, ceramides or other materials secreted by cells of the donor tissue.

32. A method for identifying transplanted micrografts at a tissue site, comprising:
applying fluorescent polymer microspheres upon a surface of an adhesive drape;
contacting the surface of the adhesive drape to donor tissue micrografts to adhere the micrografts to the surface;
applying the surface of the adhesive drape to the tissue site to allow migration of the donor tissue to the tissue site;
directing a light source onto the tissue site to visualize a location of the micrografts.

33. The method of claim 32, wherein the marker is detected by visual or ultraviolet light.

34. The method of claim 32, wherein the marker is fluorescent.

35. A method for treating an epithelial tissue wound, comprising:
harvesting donor epithelial tissue comprising cells and a cellular matrix;
contacting the donor epithelial tissue with a marker that binds to one or both of the cells and cellular matrix; and
implanting the donor epithelial tissue onto a surface of the epithelial tissue wound.

36. The method of claim 35, wherein the contacting is performed prior to the implanting.

37. The method of claim 35, wherein the harvesting comprises producing a plurality of micrografts from a donor tissue.

38. The method of claim 37, wherein the harvesting further comprises adhering the micrografts to an adhesive drape.

39. The method of claim 38, wherein the adhesive drape comprises the marker coated on a surface of the drape.

40. The method of claim 38, wherein contacting the donor epithelial tissue with the marker comprises applying the marker to the micrografts after the adhering of the micrografts to the adhesive drape.

41. The method of claim 35, further comprising visualizing the marker on the surface of the epithelial tissue wound to identify regions on the surface of the epithelial tissue wound where the donor tissue is implanted.

42. The method of claim 41, further comprising removing slough from the surface of the epithelial tissue wound, wherein the slough is removed from regions on the surface of the epithelial tissue wound other than the regions where the donor tissue is implanted.

43. The systems, apparatuses, and methods substantially as described herein.
